(19) United States
(12) Patent Application Publication
(10) Pub. No.: US 2009/0182238 A1
(43) Pub. Date: Jul. 16, 2009

(54) Method of predicting a blood pressure trend by blood pressure measurements

(75) Inventor: Kuo-Liang Chen, Taipei County (TW)

Correspondence Address: BACON & THOMAS, PLLC, 625 SLATERS LANE, FOURTH FLOOR, ALEXANDRIA, VA, 22314-1176, US

(73) Assignee: TRIPLE PRECISION INT'L. CO., LTD, Lujhou City (TW)

(21) Appl. No.: 12/007,621

(22) Filed: Jan. 14, 2008

(51) Int. Cl. A61B 5/021

(52) U.S. Cl. 600/485

(57) ABSTRACT

The present invention discloses a method of predicting a blood pressure trend by blood pressure measurements, and the method allows users to select a number of times for measuring blood pressures as a cycle according to a doctor's recommendation or a personal preference, and automatically calculates and stores an average of blood pressures of the cycle (including systolic and diastolic blood pressures). With the variation of average blood pressures of previous and next cycles, users can know about the variation of the trends of their systolic and diastolic blood pressures quickly and easily. The trend of blood pressures is provided as a reference for users to adjust their living habits and for medical professionals to diagnose a patient's condition in hope of resuming the normal blood pressure of the patient.

### BACKGROUND OF THE INVENTION

1. Field of the Invention

The present invention relates to a method of predicting a blood pressure trend by blood pressure measurements, and more particularly to a method of predicting a blood pressure trend by a quick reference to the variation of blood pressures in a number of times (or a cycle) of specific previous and next measurements.

2. Description of the Related Art

The measurement of blood pressures is an important index for analyzing cardiovascular diseases, and a risk analysis can be conducted according to the readings of blood pressures to effectively prevent cardiovascular diseases including stroke, arrhythmia and myocardial infarction, etc, and thus it is very useful to understand the effect of a relation between the time (or our living habits) and a variation of blood pressures to the risk analysis of the diseases of our cardiovascular system.

At pressure, electronic sphygmomanometers for measuring blood pressures at home become increasingly popular, and these sphygmomanometers generally adopt a buzzer or a display device to inform users about a single measurement of blood pressure that exceeds a predetermined standard value. However, the aforementioned prior art still has a drawback, since the method uses a single measurement of blood pressure as a reference to determine whether or not the blood pressure exceeds a standard value, but it does not provide the information of whether the blood pressure within a certain period of time is controlled properly or the blood pressure keeps rising.

### SUMMARY OF THE INVENTION

In view of the foregoing shortcoming of the prior art, the inventor of the present invention based on years of experience in the related industry to conduct extensive experiments and researches, and finally invented a method of predicting a blood pressure trend by several blood pressure measurements to overcome the shortcoming of the prior art.

It is a primary objective of the present invention to provide a method of predicting a blood pressure trend by blood pressure measurements, and the method allows users to select recording a certain number of blood pressure measurements as a cycle. When the number of blood pressure measurements is accumulated to the predetermined cycle, an average of blood pressures of the cycle will be calculated and stored automatically (wherein the method of calculating the average is a general average method or a weighted average method.) If a next cycle of blood pressure measurement is recorded, an average of blood pressures of the next cycle will be calculated and stored automatically, so that users can quickly know about the variation of the trend of their systolic and diastolic blood pressures by the variation of blood pressures between previous and next cycles. The trend is provided as a reference for users to improve their living and diet habits, and for medical professionals to provide appropriate medications and treatments to patients, so as to achieve the effect of preventing risks.

Another objective of the present invention is to allow users to change the accumulated number of times (the numeric value of the cycle) of the blood pressure measurements anytime, so that when a user selects examining different measuring cycles, the variation of the trend of blood pressures in different measuring cycles allows the user to know whether or not the blood pressures are controlled properly, and whether or not their living and diet habits require further adjustments.

A further objective of the present invention is to examine a variation of blood pressures of a cycle within a specific time period such as a comparison of the trends of blood pressures in the same month of different years, or in different seasons of the same year.

To achieve the foregoing objectives, the present invention provides an electronic sphygmomanometer that comprises a microprocessor module and a memory module. The microprocessor module is provided for controlling the operation of the whole electronic sphygmomanometer, wherein the memory module is provided for storing the values of the measured blood pressures, so that users can set the numeric value of a cycle to examine the variation of blood pressures in successive cycles in order to know about the variation of the trend of their systolic and diastolic blood pressures.

To make it easier for the examiner to understand the object, shape, structure, device, characteristics and functions of this invention, the specification accompanied by the drawings is described as follows.

### BRIEF DESCRIPTION OF THE DRAWINGS

FIG. 1 shows a schematic view of an apparatus corresponding to the process of the present invention, and

FIG. 2 shows a flow chart of the present invention.

### DETAILED DESCRIPTION OF THE PREFERRED EMBODIMENTS

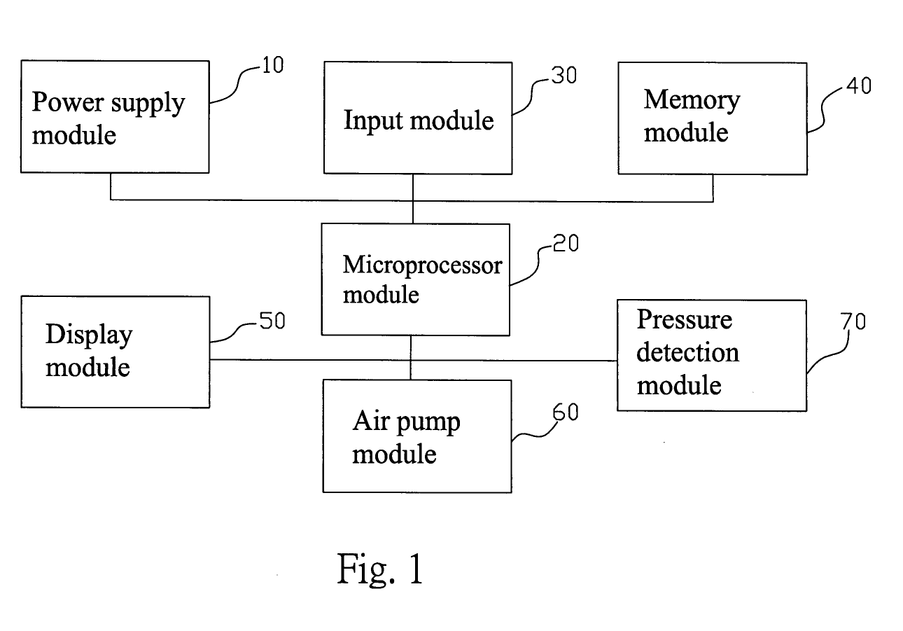

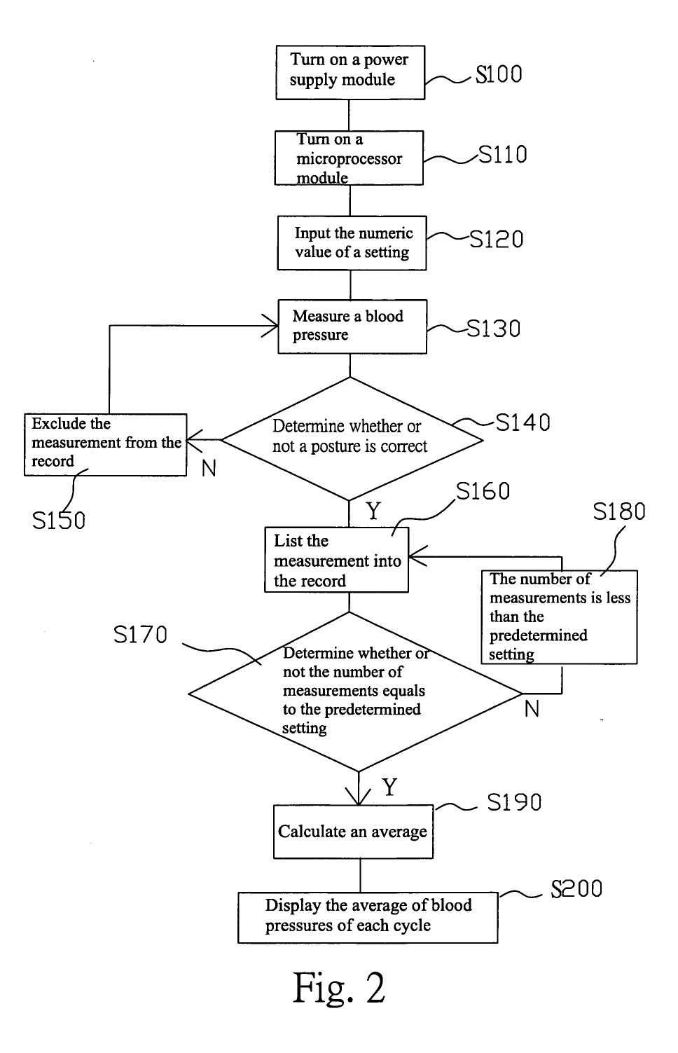

Referring to FIGS. 1 and 2 for a method of predicting a blood pressure trend by several blood pressure measurements, the method comprises the following steps:

Turn on a power supply module 10 of an electronic sphygmomanometer (S100), so that the power supply module 10 supplies the required power to a microprocessor module 20 connected to the power supply module 10 and set the microprocessor module 20 to an operating mode (S110).

A user sets the numeric value of a cycle through an input module 30, and the numeric value of the cycle is a cumulative number of times of measuring blood pressures. For example, the number of times of measurements is set to 10, 20, or 30, and the set numeric value of the cycle is stored into a memory module 40 (S120).

Measure a blood pressure (S130).

The microprocessor module 20 determines whether or not the measurement at the time complies with the standard, when the measurement of blood pressures is performed each time. If the measurement is affected by incorrect posture, coughing or other factors, then the accuracy of measuring blood pressures will be affected (S140).

If the measurement at the time does not comply with the standard, then the measurement at the time will not be listed into the record (S150), and another measurement will be taken (S130);

If the measurement at the time complies with the standard, then the measurement at the time will be listed into the record (S160) and stored into the memory module 40.

The number of times of measurements is accumulation by a counter, and a predetermined numeric value of setting is read from the memory module 40 and compared with the numeric value of the counter (S170).

If the predetermined numeric value of setting has not been reached, then the measured result at the time will be recorded and stored into the memory module 40 (S180).

Until the number of times of measurements has reached the predetermined numeric value of setting for the cycle, the microprocessor module 20 will retrieve all measured results at the time from the memory module 40, and calculate an average, and then store the average of blood pressures at the time into the memory module 40 (S190).

Users can know about the variation of the trend of their blood pressure easily and quickly to facilitate users to adjust their living habits or provide medical professionals a reference to diagnose according to the information of average blood pressures.

Further, users can use an input module 30 to change the numeric value of the cycle, such as increasing or decreasing the number of times in a cycle and allow users to know about the variation of a blood pressure trend in cycles with different number of times of measurements.

In addition, users can select the cycle of measuring blood pressures within a time period through the input module 30. For example, a variation of cycle of measuring blood pressures in the same month of different years (or the same period of last year and this year), or a variation of cycle for measuring blood pressures in different seasons of the same year can be selected.

By the records of blood pressure measurements, and the selection of a variation of average blood pressures within cycles of different number of measurements or specific time periods at different time, we can determine whether or not the blood pressure is controlled properly, so as to make preparation or adjust our living habits in advance.

To cope with the aforementioned method, the invention provides an electronic sphygmomanometer as shown in FIG. 1, and the electronic sphygmomanometer includes a microprocessor module 20, and the microprocessor module 20 includes a processor which is a microprocessing unit (MCU) to facilitate controlling the operations of the whole electronic sphygmomanometer, and the microprocessor module 20 is coupled to a power supply module 10, an input module 30, a memory module 40, an air pump module 60, a pressure detection module 70 and a display module 50, wherein the air pump module 60 is provided for pressurization, and the air pump module 60 includes a release valve for releasing a small amount of air during the blood pressure is being measured or releasing the air quickly after the blood pressure measurement is taken. The pressure detection module 70 is provided for detecting a variation of blood pressures and transmitting the variation of blood pressures to the microprocessor module 20. The display module 50 is provided for outputting related numeric values of the measured results, and the display module 50 of this embodiment is a display device. The memory module 40 is provided for storing the measured value of blood pressures for each time or an average of blood pressure of a cycle. The input module 30 is provided for users to operate, set and change the number of times in a cycle and provided as an interface for transmitting data.

In summation of the above description, the method of predicting a blood pressure trend by blood pressure measurements in accordance with the present invention enhances the performance than the conventional structure and further complies with the patent application requirements and is submitted to the Patent and Trademark Office for review and granting of the commensurate patent rights.
S100 Turn on a power supply module.
S110 Turn on a microprocessor module.
S120 Input the numeric value of a setting.
S130 Measure a blood pressure.
S140 Determine whether or not a posture is correct.
S150 Exclude the measurement from the record, if the posture is incorrect.
S160 List the measurement into the record, if the posture is correct.
S170 Determine whether or not the number of measurements equals to the predetermined setting.
S180 Record the measured result of this time, if the number of measurements is less than the predetermined setting.
S190 Calculate an average of all measured results within the predetermined setting and store the average into a memory module, if the number of measurements is equal to the predetermined setting.
S200 Set the numeric value as a cycle, and show the average of blood pressure of each cycle on a display module 50, so that users can examine whether or not their blood pressure is rising or maintained stable from the variation of average blood pressures of the cycles.
10 Power supply module
20 Microprocessor module
30 Input module
40 Memory module
50 Display module
60 Air pump module
70 Pressure detection module

FIG. 1
S100 Turn on a power supply module.
S110 Turn on a microprocessor module.
S120 Input the numeric value of a setting.
S130 Measure a blood pressure.
S140 Is the posture correct? Yes/No
S150 Exclude the measurement from the record.
S160 List the measurement into the record.
S170 Is the number of measurements equal to the predetermined setting? Yes/No
S180 The number of measurements is less than the predetermined setting.
S190 Calculate an average.
S200 Display the average of blood pressures of each cycle.

FIG. 2

What is claimed is:

1. A method of predicting a blood pressure trend by blood pressure measurements, comprising the steps of:
S1: setting a predetermined cumulative number of measurements as a cycle;
S2: recording a blood pressure measurement for each time of the cycle;
S3: determining whether or not said number of measuring blood pressure reaches said predetermined cumulative number of measurements, if yes, then go to S4, or else go to S2;
S4: calculating and storing an average (systolic and diastolic pressures) of said cycle; and
S5: performing S2 to record the blood pressure measurements and the average of a cycle;
thereby, users can easily and quickly know about the variation of trends of their blood pressures of a cycle by the average blood pressure of a cycle, so as to facilitate uses to adjust their living habits and provide a reference for medical professionals to diagnose according to said average blood pressure.

2. The method of predicting a blood pressure trend by several blood pressure measurements according to claim 1, further comprising a step of changing the numeric value of said cycle by users.

3. The method of predicting a blood pressure trend by several blood pressure measurements according to claim 2, further comprising a step of displaying successive blood pressure averages after said cycle is changed.

4. The method of predicting a blood pressure trend by several blood pressure measurements according to claim 2, further comprising a step of displaying a variation of cycles of blood pressured within a specific period according to a user's requirement.

5. The method of predicting a blood pressure trend by several blood pressure measurements according to claim 4, wherein said specific period is the same specific time interval of the same year.

6. The method of predicting a blood pressure trend by several blood pressure measurements according to claim 4, wherein said specific time is a different season of the same year.

7. The method of predicting a blood pressure trend by several blood pressure measurements according to claim 6, wherein said specific season is spring, summer, fall or winter.

8. The method of predicting a blood pressure trend by several blood pressure measurements according to claim 1, comprising the steps of:
turning on a power supply switch of an electronic sphygmomanometer;
setting a microprocessor module at an operating mode;
inputting a predetermined numeric value of setting through an input module, and storing said predetermined numeric value of setting into a memory module;
performing a measurement; and
storing an average of blood pressures at the time into said memory module, after said microprocessor module retrieves all measured results within the number of times of measurements from said memory module and calculates an average, until said numeric value of measurements equals to said predetermined numeric value of setting.

9. The method of predicting a blood pressure trend by several blood pressure measurements according to claim 1, wherein said microprocessor module determines whether or not said measurement at the time complies with the standard to prevent incorrect posture, coughing or other factors affecting said measurement and causing an inaccurate measurement at the time, when a measurement is performed each time;
said microprocessor does not list said measurement at the time in a record, and performs another measurement, if said measurement at the time does not comply with the standard; and
said microprocessor lists said measurement at the time in a record, if said measurement at the time does not comply with the standard.

10. The method of predicting a blood pressure trend by several blood pressure measurements according to claim 1, wherein a counter is provided for accumulating the number of times, and said memory module is provided for reading and comparing said predetermined numeric value of setting with a numeric value of said counter after a measurement is completed each time; and said measured result at the time is recorded and stored into said memory module, if said predetermined numeric value of setting is not reached.
